(19) United States
(12) Patent Application Publication
(10) Pub. No.: US 2020/0345552 A1
(43) Pub. Date: Nov. 5, 2020

(54) ILLUMINATION VARIABILITY REDUCTION IN LASER TREATMENT SYSTEMS

(71) Applicant: Xinova, LLC, Seattle, WA (US)

(72) Inventor: Seth Adrian MILLER, Longmont, CO (US)

(73) Assignee: Xinova, LLC, Seattle, WA (US)

(21) Appl. No.: 16/765,150

(22) PCT Filed: Nov. 28, 2017
(86) PCT No.: PCT/US17/63518

§ 371 Date: May 18, 2020

(51) Int. Cl. A61F 9/008

(52) U.S. Cl. CPC A61F 9/00823; A61B 2018/00595; A61F 2009/00863

(57) ABSTRACT

Technologies are provided for laser treatment with reduced illumination variability. In some examples, a laser beam that creates a first illumination variation at a target site may be adjusted such that the laser beam orientation changes slightly while the laser beam is still being generated. The adjusted laser beam may create a second illumination variation at the target site, and because the laser beam orientation is different the first and second illumination variations may combine to form a combined illumination variation at the target site whose variability is less than the variability of the first or second illumination variations. The more-uniform combined illumination variation may then be used to treat the target site, for example via heat generation.

### BACKGROUND

Unless otherwise indicated herein, the materials described in this section are not prior art to the claims in this application and are not admitted to be prior art by inclusion in this section.

Lasers can be used to treat certain diseases. For example, eye diseases may be treated via a coagulation mechanism in which blood vessels within the eye are cauterized using energy from a laser. Laser treatment may be effective for diseases such as diabetic retinopathy, diabetic macular edema, macular degeneration, or other retinopathy. Such laser treatments may require precise and accurate application of laser energy in order to provide effective treatment yet avoid unintentional damage of the treatment area.

### SUMMARY

The present disclosure generally describes techniques to reduce illumination variability in laser treatment systems.

According to some examples, a method for laser treatment with increased illumination uniformity is provided. The method may include generating a laser beam to create a first illuminated region at a first location of a target site and adjusting the laser beam to create a second illuminated region at a second location of the target site, where the first and second regions at least partially overlap to form a combined illuminated region at the target site. A uniformity of illumination of the combined illuminated region at the target site may be greater than a uniformity of illumination of the first illuminated region at the target site. The method may further include performing treatment at the target site using the combined illuminated region of the laser beam.

According to other examples, a laser treatment system is provided. The system may include a laser configured to generate a laser beam to create a first illuminated region at a first location of a target site and an adjustment device coupled to the laser and configured to adjust the laser beam to create a second illuminated region at a second location of the target site. The system may further include a controller coupled to the laser and the adjustment device and configured to control an operation of the adjustment device such that the first and second regions at least partially overlap to form a combined illuminated region at the target site and a uniformity of the illumination of the combined illuminated region at the target site is greater than a uniformity of illumination of the first illuminated region at the target site.

According to further examples, another laser treatment system is provided. The system may include a laser configured to generate a laser beam to create a first illuminated region at a first location of a target site and generate heat at the target site through the laser beam and an electro-optic device having an adjustable refractive index medium, coupled to the laser, and configured to oscillate the laser beam in a first direction to create a second illuminated region at a second location of the target site. The system may further include a controller coupled to the laser and the electro-optic device and configured to control an operation of the electro-optic device such that the first and second regions at least partially overlap to form a combined illuminated region at the target site, where a uniformity of illumination of the combined illuminated region at the target site is greater than a uniformity of illumination of the first illuminated region at the target site.

According to yet further examples, a method for reducing laser illumination variability is provided. The method may include generating a laser beam directed at a substrate, where the laser beam creates a first illumination variation on the substrate. The method may further include deflecting the laser beam to create a second illumination variation on the substrate, where a combined illumination variation formed from the first and second illumination variations has a lower average variation than either the first or second illumination variations. The method may further include using the laser beam forming the combined illumination variation to generate heat on the substrate.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 illustrates illumination variability in a laser system;

FIG. 2 illustrates an example laser system with reduced illumination variability;

FIG. 3 illustrates an example electro-optic device that may be used to reduce illumination variability in a laser system;

FIG. 4 illustrates how illumination variability in a laser system may be reduced by wobbling;

FIG. 5 illustrates a general purpose computing device, which may be used to reduce illumination variability in a laser treatment system;

FIG. 6 is a flow diagram illustrating an example method to reduce illumination variability in laser treatment systems that may be performed by a computing device such as the computing device in FIG. 5; and

FIG. 7 illustrates a block diagram of an example computer program product, some of which arranged in accordance with at least some embodiments described herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

This disclosure is generally drawn, inter alia, to methods, apparatus, systems, devices, and/or computer program products related to reduction of illumination variability in laser treatment systems.

Briefly stated, technologies are generally described for laser treatment with reduced illumination variability. In some examples, a laser beam that creates a first illumination variation at a target site may be adjusted such that the laser beam orientation changes slightly while the laser beam is still being generated. The adjusted laser beam may create a second illumination variation at the target site, and because the laser beam orientation is different the first and second illumination variations may combine to form a combined illumination variation at the target site whose variability is less than the variability of the first or second illumination variations. The more-uniform combined illumination variation may then be used to treat the target site, for example via heat generation.

Laser ophthalmology systems rely on the precise and accurate delivery of laser energy to treat eye diseases. For example, some laser ophthalmology systems may perform treatment by using lasers to generate heat in a portion of the eye, such as the retinal pigment epithelium (RPE) layer. To ensure effective treatment, such systems may need to be configured to deliver enough energy to a target area, and with sufficient spatial uniformity, in order to achieve the desired treatment goal (for example, to generate heat in tissue and/or to cauterize blood vessels) within the target area. However, mechanical imperfections and/or contaminants within the laser system may affect laser energy delivery, uniformity, and/or targeting.

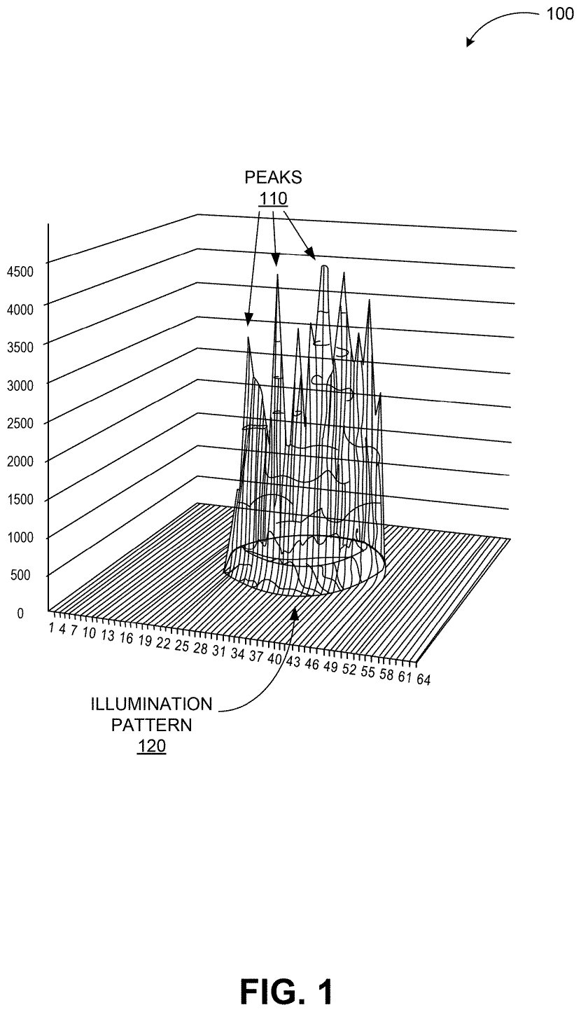

FIG. 1 illustrates illumination variability in a laser system. Diagram 100 in FIG. 1 depicts illumination variations in a laser beam incident on a surface over a time duration, where the horizontal axes represent physical coordinates on the surface and the vertical axis represents illumination or energy. As diagram 100 depicts, the illumination pattern 120 cast by a laser beam on a surface may vary across the surface. In particular, the illumination pattern 120 includes illumination peaks 110, where the illumination is greater than the average illumination over the entire pattern, as well as locations where the illumination is less than the average illumination. Such illumination variations, also referred to as “speckle”, may be caused by physical imperfections in the laser system, or by the presence of dust or particulates on or near optical elements (for example, lenses, mirrors, etc.) in the system. The extent of illumination variation for a particular illumination pattern may be characterized in any suitable way. In one embodiment, illumination variation may be represented by a ratio of the illumination or intensity of the largest illumination peak to the average illumination or intensity of the entire illumination pattern.

In laser treatment systems, excessive illumination variations or speckle can be problematic because they make it difficult to determine whether the appropriate amount of energy has been delivered to a desired target location or treatment site. Too much energy (for example, at locations where the peaks 110 are present) may result in over-treatment and undesired damage, but insufficient energy (for example, at locations where illumination is relatively low) may result in under-treatment. When speckle is present, controlling laser treatment to limit the size of illumination peaks may result in significant under-treatment, controlling laser treatment to limit the minimum illumination may result in significant over-treatment, and controlling laser treatment based on average illumination may result in some over-treatment and some under-treatment. Accordingly, reducing illumination variability or speckle in laser treatment systems may increase illumination uniformity and reduce both over-treatment and under-treatment. While example embodiments are described herein using pulsed laser therapy techniques, similar approaches may also be used with continuous wave (CW) laser. A timing of the applied CW laser may be determined to generate (but not over-generate) sufficient heat at the treatment site while speckle pattern may be generated using the principles described herein.

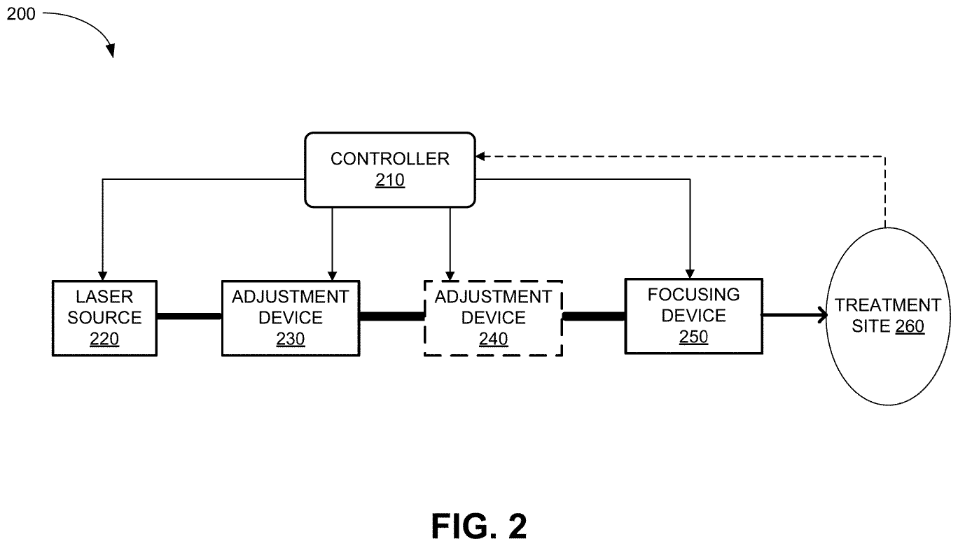

FIG. 2 illustrates an example laser system 200 with reduced illumination variability, arranged in accordance with at least some embodiments described herein.

Laser system 200 may include a laser source 220 and one or more adjustment device(s) 230/240 within the optical path of the laser source 220 and configured to adjust a laser beam from the laser source 220 to reduce illumination variations, as described below. Laser system 200 may further include a focusing device 250 within the optical path of the adjustment device(s) 230/240, configured to adjust the focus of the laser beam output from the adjustment device(s) 230/240 on a treatment site 260, which may be a portion of a retinal pigment epithelium (RPE) layer. Laser system 200 may also include a controller 210 configured to control and adjust laser source 220, adjustment devices 230/240, and focusing device 250, based on sensor inputs received from the treatment site 260 and inputs received from other sources.

The laser source 220 may be configured to generate a continuous-wave laser beam, or may be configured to generate laser beam pulses of a given duration. In some embodiments, the laser source 220 may be configured to generate laser beam pulses with duration less than or equal to two microseconds. The laser source 220 may generate a laser beam of any wavelength or frequency suitable for the application at hand (for example, treatment of an eye disease). Similarly, the laser source 220, in conjunction with the focusing device 250, may generate a laser beam that creates an illuminated region at the treatment site 260 with any size suitable for the application at hand. For example, the laser source 220 and the focusing device 250 may generate a laser beam that creates, at the treatment site 260, an illuminated region with a diameter less than 50 μm, less than 70 μm, or with any suitable diameter. Assuming a wavelength of the laser light to be about 0.5 μm and the spot size about 50 μm, a minimum practical overlap range for the first and second illumination regions may be about 5% (may also be closer to about 10% depending on practical implementation conditions). A maximum overlap may be assumed about 50%, both to preserve the spot size and prevent the system from generating two spots. Thus, an overlap range of 5% to 50% may be a reasonable range for a system according to embodiments.

Laser beams (pulses) typically include a plurality of intensity spikes. Overall, a laser beam may have a Gaussian-like profile underneath the speckle. Thus, a laser beam radius (or beam width or spot size) may be defined in various ways, such as full width at half maximum (FWHM). Because speckle may raise the maximum intensity substantially, laser beam width may be defined in terms of a smoothed maximum, or as a lower threshold (e.g., 10% or 20%) of the maximum intensity. The smoothing range may be characterized as greater than a spike size, for example, in a range of 2% to 20%, which may correspond to 1-10 μm for a 50 μm laser spot.

The adjustment device(s) 230/240 may be configured to adjust the laser beam from the laser source 220 to reduce illumination variations in the region illuminated by the laser beam at the treatment site 260. In one embodiment, the adjustment device(s) 230/240 may adjust the laser beam by deflecting the laser beam in a periodic fashion, also referred to as “wobbling” or “oscillating” the laser beam. “Oscillating” a laser beam, in the context of this disclosure, may refer to adjusting, in a periodic or aperiodic fashion, a path or angle of the laser beam, or some parameter associated with the generation or propagation of the laser beam to affect the laser beam path. For example, the oscillation may be sinusoidal in nature or may occur in discrete steps. “Oscillation”, which may refer to the change in some parameter, may be used to describe the deflection of the laser beam, an angle-of-deflection of the laser beam, variations in a position of incidence of the laser beam on a medium, substrate, or target site, a control signal applied to the adjustment device(s) 230/240, or any other suitable parameter.

In some embodiments, the laser beam (pulses) may be “oscillated” during the pulse treatment time. Thus, the oscillation may be considered as the oscillation of a CW laser beam in a very short time period (during each pulse). In practical implementations, the time period may be in microseconds (μs). In example implementations, where a smoothed composite pulse is being created, the laser pulse may be directed towards multiple locations (two or more).

In one embodiment, the adjustment device 230 may be configured to deflect the laser beam along a first axis or direction, and the adjustment device 240 may be configured to deflect the laser beam along a second axis or direction least partially orthogonal to the first axis or direction. The undeflected laser beam may create a first illuminated region at the treatment site 260 with a particular illumination variation or speckle pattern. The deflected laser beam may also create a second illuminated region at the treatment site 260 that has the same illumination variation or speckle pattern as the undeflected laser beam, but because the deflected laser beam is directed at a slightly different location at the treatment site 260 the second illuminated region may at least partially differ from the first illuminated region. The combination of the first and second illuminated regions, which may be referred to as a combined illuminated region, may have an illumination variation that, on average, is less than the illumination variations of either the first illuminated region (from the undeflected laser beam) or the second illuminated region (from the deflected laser beam). The combined illuminated region may be larger than either the first or second illuminated regions, and may be based on the degree to which the laser beam is deflected and the distance between the deflector(s) (for example, the adjustment device(s) 230/240) and the treatment site 260.

In one embodiment, the diameter of the first and second illuminated regions may be less than or equal to about 50 μm, the distance between the deflector(s) and the treatment site 260 may be greater than or equal to 10 centimeters (cm), the deflector(s) may be configured to deflect the laser beam with a maximum total deflection angle of 0.1 milliradians (mrad), such that the laser beam is displaced laterally by about 30 μm or less and the diameter of the combined illuminated region may be less than or equal to about 70 nm. After deflection by the adjustment device(s) 230/240, the laser beam may pass through the focusing device 250, which may be configured (for example, via the controller 210) to focus the laser beam to achieve a desired size or diameter for the combined illuminated region at the treatment site 260. For example, the focusing device 250 may be configured to focus the laser beam to achieve a combined illuminated region diameter of 70 micrometers or less. In some embodiments, a ratio of the diameter of the first (or second) illuminated region and the diameter of the combined illuminated region may range from about 1:1 to about 1:10.

As described above, adjustment devices such as the adjustment device(s) 230/240 may be used to deflect a laser beam to reduce illumination variations. Accordingly, adjustment devices may be selected based on the characteristics of the laser beam to be deflected. For example, an adjustment device configured to deflect a laser beam relatively quickly may be used to deflect a relatively-short-duration laser beam, whereas an adjustment device that can only deflect a laser beam relatively slowly may be used to deflect a laser beam with relatively long duration. Adjustment devices may deflect a laser beam using any suitable mechanism. In some embodiments, adjustment devices may use opto-electric interference, opto-acoustic interference, and/or mechanical interference to deflect laser beams.

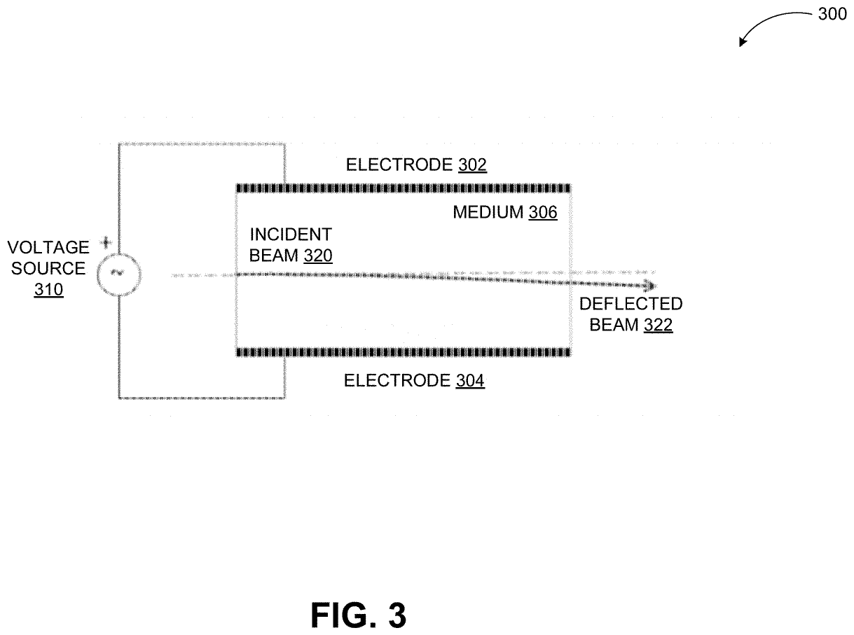

An adjustment device that uses an opto-electric interference mechanism to deflect a laser beam may use electrical potentials and/or currents to interfere with and cause an incident laser beam to change direction. For example, as described in more detail in FIG. 3, an opto-electric interference mechanism may be implemented using an adjustable refractive index medium, where the refractive index of the medium varies based on an applied electric potential. Adjusting an electric potential applied to the medium may adjust the refractive index of the medium, which in turn may deflect the path of a laser beam passing through the medium.

An adjustment device that uses opto-acoustic interference to deflect a laser beam may use acoustic signals to interfere with and cause an incident laser beam to change direction. For example, an opto-acoustic interference mechanism may be implemented using a medium that vibrates due to an applied acoustic signal (for example, via a piezoelectric transducer). When the medium vibrates, its refractive index may change, which may deflect the path of a laser beam passing through the medium.

An adjustment device that uses mechanical interference to deflect a laser beam may use a mechanically-actuated physical structure to interfere with and cause an incident laser beam to change direction. For example, a mechanical interference mechanism may be implemented using a mechanically-actuated reflective surface, lens, or other medium. The mechanical actuation (for example, rotation, displacement, vibration, etc.) of the medium may deflect the path of a laser beam passing through or reflecting from the medium.

FIG. 3 illustrates an example electro-optic device 300 that may be used to reduce illumination variability in a laser system, arranged in accordance with at least some embodiments described herein.

The electro-optic device 300, which may be or integrated into an adjustment device similar to the adjustment devices 230/240, may include two electrodes 302 and 304, positioned on opposite sides of a medium 306, similar to the configuration of a capacitor. The electrodes 302 and 304 may be connected to a voltage source 310 such that an electrical potential can be applied to the medium 306 through the electrodes 302 and 304. The medium 306, in turn, may have a refractive index that varies based on the applied electrical potential. When an incident laser beam 320 passes through the medium 306, the medium 306 deflects the beam, forming a deflected laser beam 322, where the extent of deflection is based on the medium refractive index, which in turn is based on the applied electrical potential. Accordingly, varying the electrical potential provided by voltage source 310 may vary the extent of deflection of the incident beam 320. In some embodiments, the electro-optic device may include a Pockels cell whose medium includes a crystal mixture of potassium tantalite and potassium niobate (also referred to as “KTN”), although any electro-optic device having a medium whose refractive index varies based on an applied electrical potential may be used.

As described above, a laser beam may be “wobbled” or “oscillated”, which may mean deflecting the laser beam in a periodic fashion, to reduce illumination variability in the illuminated region at a treatment site. The periodicity (or alternately frequency) of the wobble or oscillation may be selected based on the duration of the treatment and/or the laser beam generation. In embodiments where the generated laser beam is a pulse having a duration less than or equal to two microseconds, the beam wobbling or oscillating may be configured to occur at a frequency greater than or equal to 0.6 megahertz (MHz), to enable the beam deflection to occur during the pulse.

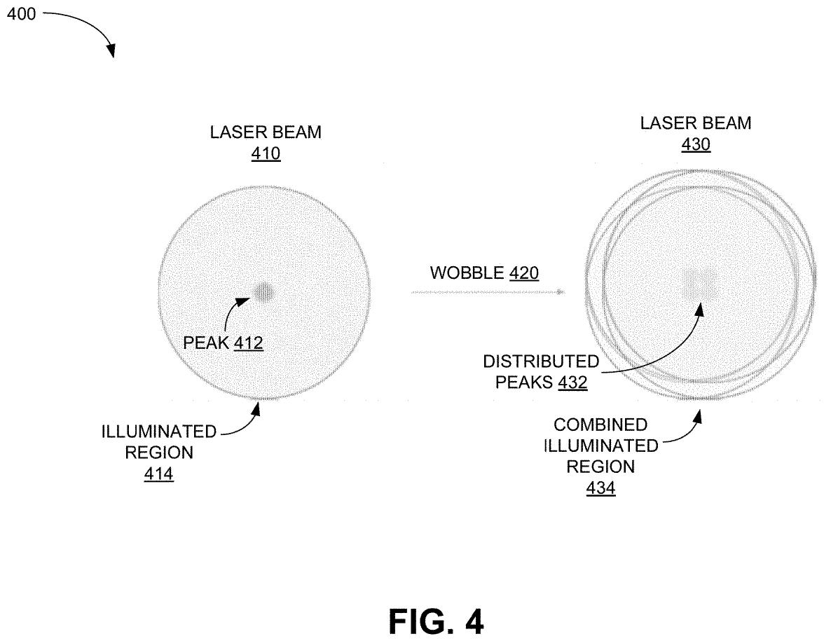

FIG. 4 illustrates how illumination variability in a laser system may be reduced by wobbling, arranged in accordance with at least some embodiments described herein.

In diagram 400, a laser beam 410 may generate an illuminated region 414 at a treatment site that may have an illumination peak 412, similar to the peaks 110 in FIG. 1. Upon introducing a wobble 420, a resulting laser beam 430 may generate a combined illuminated region 434 that is larger than the illuminated region 414. In the combined illuminated region 434, the peak 412 may be spatially distributed or smeared into distributed peaks 432, where each of the distributed peaks may have a lower illumination intensity than the peak 412. Accordingly, the illumination variation of the combined illuminated region 434 may be less than the illumination variation of the illuminated region 414. Said another way, the uniformity of illumination of the combined illuminated region 434 may be greater than the uniformity of illumination of the illuminated region 414.

While the uniformity of illumination may be defined based on uniform heating of the treatment area, another example definition may be “speckle factor”, which is maximum intensity over average intensity. Because of noise, a threshold may be applied to the maximum intensity value in some examples. In an example implementation, the data of a laser beam analyzer may be a 2-dimensional data set with “a” (value of the radiant exposure for each position x, y) being defined as:
a(k)=value of a single pixel at position (x,y)
   To determine the “speckle factor”, the maximum radiant exposure peak “a(peak)” within the laser beam profile may be divided by average radiant exposure of the laser beam profile. To reduce noise level, data above a threshold “a(threshold)” may be considered for the data set “N” such that:
Nϵ{a(k)>a(threshold)}
   With an index “k” for each element in the data set “N” and the cardinality of “N” as “|N|”, the speckle factor may be defined as:

$$\frac{a ( peak )}{{Σ}_{k} \frac{N ( k )}{\vert N \vert}}$$

Laser beam illumination variability may also (or instead) be adjusted in other ways besides beam deflection. In some embodiments, a laser beam's beam profile or optical path may be adjusted, which in turn may change the speckle pattern and therefore change the illumination variability of the laser beam. In other embodiments, a laser beam may be rotated (for example, along an axis parallel to the laser beam) instead of deflected to adjust its illumination variability.

While the above description is couched in the context of laser treatment systems for eye diseases, the illumination variability reduction techniques described herein may also be applicable to other laser systems. For example, the techniques described herein may be used to reduce illumination variability on any suitable substrate, thereby increasing illumination and heating uniformity on the substrate.

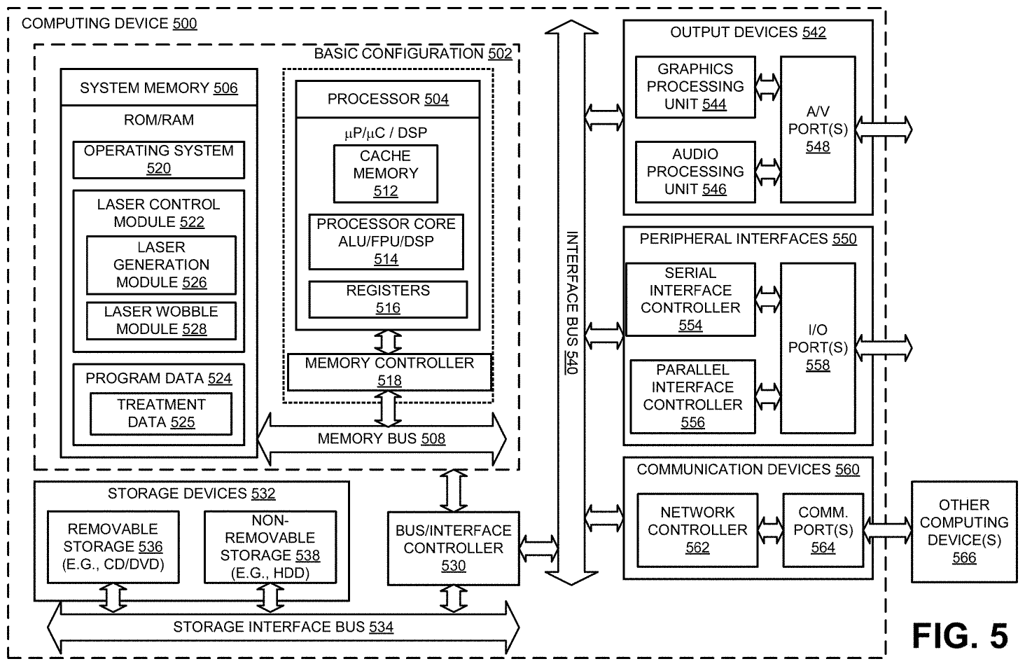

FIG. 5 illustrates a computing device, which may be used to reduce illumination variability in a laser treatment system, arranged in accordance with at least some embodiments described herein.

For example, a computing device 500 may be used to control the deflection of a laser beam in a laser treatment system as described herein. In an example basic configuration 502, the computing device 500 may include one or more processors 504 and a system memory 506. A memory bus 508 may be used to communicate between the processor 504 and the system memory 506. The basic configuration 502 is illustrated in FIG. 5 by those components within the inner dashed line.

Depending on the desired configuration, the processor 504 may be of any type, including but not limited to a microprocessor (μP), a microcontroller (μC), a digital signal processor (DSP), or any combination thereof. The processor 504 may include one or more levels of caching, such as a cache memory 512, a processor core 514, and registers 516. The example processor core 514 may include an arithmetic logic unit (ALU), a floating point unit (FPU), a digital signal processing core (DSP Core), or any combination thereof. An example memory controller 518 may also be used with the processor 504, or in some implementations, the memory controller 518 may be an internal part of the processor 504.

Depending on the desired configuration, the system memory 506 may be of any type including but not limited to volatile memory (such as RAM), non-volatile memory (such as ROM, flash memory, etc.) or any combination thereof. The system memory 506 may include an operating system 520, a laser control module 522, and program data 524. The laser control module 522 may include a laser generation module 526 configured to control a laser source and a laser wobble module 528 configured to wobble or oscillate a laser beam to reduce laser illumination variability as described herein. The program data 524 may include, among other data, treatment data 525 associated with desired laser treatment parameters, or the like.

The computing device 500 may have additional features or functionality, and additional interfaces to facilitate communications between the basic configuration 502 and any desired devices and interfaces. For example, a bus/interface controller 530 may be used to facilitate communications between the basic configuration 502 and one or more data storage devices 532 via a storage interface bus 534. The data storage devices 532 may be one or more removable storage devices 536, one or more non-removable storage devices 538, or a combination thereof. Examples of the removable storage and the non-removable storage devices include magnetic disk devices such as flexible disk drives and hard-disk drives (HDDs), optical disk drives such as compact disk (CD) drives or digital versatile disk (DVD) drives, solid state drives (SSDs), and tape drives to name a few. Example computer storage media may include volatile and nonvolatile, removable and non-removable media implemented in any method or technology for storage of information, such as computer readable instructions, data structures, program modules, or other data.

The system memory 506, the removable storage devices 536 and the non-removable storage devices 538 are examples of computer storage media. Computer storage media includes, but is not limited to, RAM, ROM, EEPROM, flash memory or other memory technology, CD-ROM, digital versatile disks (DVDs), solid state drives (SSDs), or other optical storage, magnetic cassettes, magnetic tape, magnetic disk storage or other magnetic storage devices, or any other medium which may be used to store the desired information and which may be accessed by the computing device 500. Any such computer storage media may be part of the computing device 500.

The computing device 500 may also include an interface bus 540 for facilitating communication from various interface devices (e.g., one or more output devices 542, one or more peripheral interfaces 550, and one or more communication devices 560) to the basic configuration 502 via the bus/interface controller 530. Some of the example output devices 542 include a graphics processing unit 544 and an audio processing unit 546, which may be configured to communicate to various external devices such as a display or speakers via one or more A/V ports 548. One or more example peripheral interfaces 550 may include a serial interface controller 554 or a parallel interface controller 556, which may be configured to communicate with external devices such as input devices (e.g., keyboard, mouse, pen, voice input device, touch input device, etc.) or other peripheral devices (e.g., printer, scanner, etc.) via one or more I/O ports 558. An example communication device 560 includes a network controller 562, which may be arranged to facilitate communications with one or more other computing devices 566 over a network communication link via one or more communication ports 564. The one or more other computing devices 566 may include servers at a datacenter, customer equipment, and comparable devices.

The network communication link may be one example of a communication media. Communication media may be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR) and other wireless media. The term computer readable media as used herein may include both storage media and communication media.

The computing device 500 may be implemented as a part of a general purpose or specialized server, mainframe, or similar computer that includes any of the above functions. The computing device 500 may also be implemented as a personal computer including both laptop computer and non-laptop computer configurations.

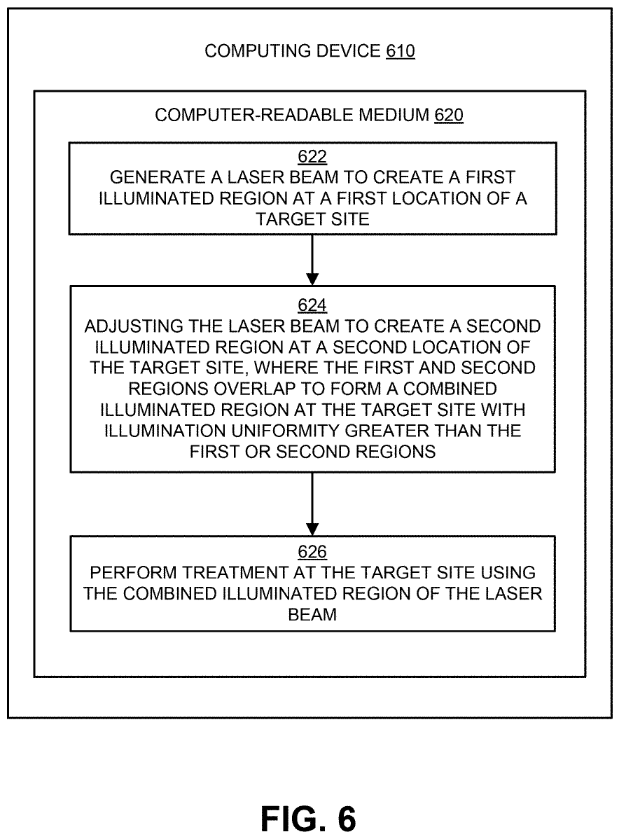

FIG. 6 is a flow diagram illustrating an example method to reduce illumination variability in laser treatment systems that may be performed by a computing device such as the computing device in FIG. 5, arranged in accordance with at least some embodiments described herein.

Example methods may include one or more operations, functions or actions as illustrated by one or more of blocks 622, 624, and/or 626, and may in some embodiments be performed by a computing device such as the computing device 600 in FIG. 6. The operations described in the blocks 622-626 may also be stored as computer-executable instructions in a tangible and non-transitory computer-readable medium such as a computer-readable medium 620 of a computing device 610.

An example process to reduce illumination variability in laser treatment systems may begin with block 622, “GENERATE A LASER BEAM TO CREATE A FIRST ILLUMINATED REGION AT A FIRST LOCATION OF A TARGET SITE”, where a laser source may generate a laser beam to illuminate a first region at a first location of a desired target site, as described above. The laser beam may be used to generate heat at the target site, for example for treatment of an eye disease, as mentioned above.

Block 622 may be followed by block 624, “ADJUSTING THE LASER BEAM TO CREATE A SECOND ILLUMINATED REGION AT A SECOND LOCATION OF THE TARGET SITE, WHERE THE FIRST AND SECOND REGIONS OVERLAP TO FORM A COMBINED ILLUMINATED REGION AT THE TARGET SITE WITH ILLUMINATION UNIFORMITY GREATER THAN THE FIRST OR SECOND REGIONS”, where the generated laser beam may be adjusted to illuminate a second region at a second location of the target site. The combination of the illuminated regions formed by the laser beam before and after adjustment may have greater illumination uniformity than either the first or second regions, as described above. The greater illumination uniformity of the combined illuminated region may result in greater uniformity of heating than either the first or second illuminated regions.

Block 624 may be followed by block 626, “PERFORM TREATMENT AT THE TARGET SITE USING THE COMBINED ILLUMINATED REGION OF THE LASER BEAM”, where the laser beam forming the combined illuminated region may be used to treat the target site. For example, in the case of a laser ophthalmology system, the laser beam may be used to heat eye tissue and/or cauterize blood vessels at the target site.

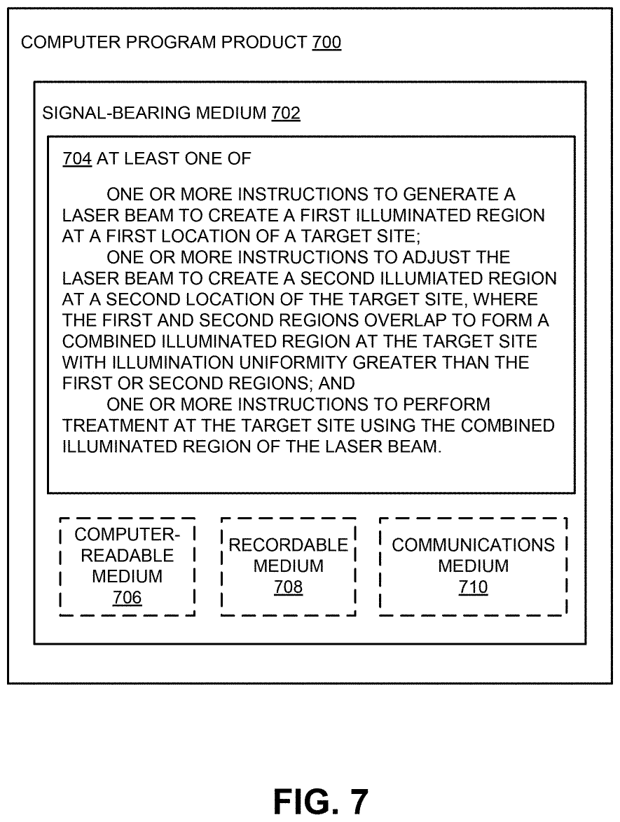

FIG. 7 illustrates a block diagram of an example computer program product, arranged in accordance with at least some embodiments described herein.

In some examples, as shown in FIG. 7, a computer program product 700 may include a signal-bearing medium 702 that may also include one or more machine readable instructions 704 that, when executed by, for example, a processor may provide the functionality described herein. Thus, for example, referring to the processor 504 in FIG. 5, the laser control module 522 may undertake one or more of the tasks shown in FIG. 7 in response to the instructions 704 conveyed to the processor 504 by the signal-bearing medium 702 to perform actions associated with reducing illumination variability in laser treatment systems as described herein. Some of those instructions may include, for example, instructions to generate a laser beam to create a first illuminated region at a first location of a target site, adjust the laser beam to create a second illuminated region at a second location of the target site, where the first and second regions overlap to form a combined illuminated region at the target site with illumination uniformity greater than the first or second regions, and/or perform treatment at the target site using the combined illuminated region of the laser beam, according to some embodiments described herein.

In some implementations, the signal-bearing medium 702 depicted in FIG. 7 may encompass tangible and non-transitory computer-readable medium 706, such as, but not limited to, a hard disk drive (HDD), a solid state drive (SSD), a compact disc (CD), a digital versatile disk (DVD), a digital tape, memory, etc. In some implementations, the signal bearing medium 702 may encompass recordable medium 708, such as, but not limited to, memory, read/write (R/W) CDs, R/W DVDs, etc. In some implementations, the signal bearing medium 702 may encompass communications medium 710, such as, but not limited to, a digital and/or an analog communication medium (e.g., a fiber optic cable, a waveguide, a wired communication link, a wireless communication link, etc.). Thus, for example, the computer program product 700 may be conveyed to one or more modules of the processor 504 by an RF signal bearing medium, where the signal-bearing medium 702 is conveyed by the wireless communications medium 710 (e.g., a wireless communications medium conforming with the IEEE 802.11 standard).

According to some examples, a method for laser treatment with increased illumination uniformity is provided. The method may include generating a laser beam to create a first illuminated region at a first location of a target site and adjusting the laser beam to create a second illuminated region at a second location of the target site, where the first and second regions at least partially overlap to form a combined illuminated region at the target site. A uniformity of illumination of the combined illuminated region at the target site may be greater than a uniformity of illumination of the first illuminated region at the target site. The method may further include performing treatment at the target site using the combined illuminated region of the laser beam.

According to other examples, adjusting the laser beam to create the second illuminated region at the second location of the target site may include oscillating the laser beam through one or more of an opto-electric interference, an opto-acoustic interference, or a mechanical interference. Oscillating the laser beam may include deflecting the laser beam to point from the first location to the second location. Oscillating the laser beam through the opto-electric interference may include oscillating the laser beam through an electro-optic device having an adjustable refractive index medium. Oscillating the laser beam through the opto-electric interference may include generating a sinusoidal variation in the laser beam through a Pockels cell device. Generating the laser beam to create the first illuminated region at the first location of the target site may include creating the first illuminated region at a portion of a retinal pigment epithelium (RPE) layer.

According to further examples, adjusting the laser beam to create the second illuminated region at the second location of the target site may include oscillating the laser beam to achieve a total deflection angle of less than 0.1 mrad for a deflector—RPE layer distance of greater than 10 centimeters. Adjusting the laser beam to create the second illuminated region at the second location of the target site may include oscillating the laser beam at a frequency of 0.6 MHz or greater. Generating the laser beam to create the first illuminated region at the first location of the target site may include generating the laser beam for a duration of less than two microseconds. Adjusting the laser beam to create the second illuminated region at the second location of the target site may include forming the combined illuminated region at a portion of the target site that has a diameter of less than 70 micrometers. The method may further include sweeping the adjusted laser beam in two orthogonal directions. The method may also include focusing the adjusted laser beam. Performing treatment at the target site using the combined illuminated region of the laser beam may include generating heat at the target site using the combined illuminated region to treat the target site.

According to some examples, a method for laser treatment with increased illumination uniformity is provided. The method may include generating a laser beam to create a first illuminated region at a first location of a target site and adjusting the laser beam to create a second illuminated region at a second location of the target site, where the first and second regions at least partially overlap at the target site. The method may further include performing treatment at the target site using the combined illuminated region of the laser beam.

According to other examples, a laser treatment system is provided. The system may include a laser configured to generate a laser beam to create a first illuminated region at a first location of a target site and an adjustment device coupled to the laser and configured to adjust the laser beam to create a second illuminated region at a second location of the target site. The system may further include a controller coupled to the laser and the adjustment device and configured to control an operation of the adjustment device such that the first and second regions at least partially overlap to form a combined illuminated region at the target site and a uniformity of the illumination of the combined illuminated region at the target site is greater than a uniformity of illumination of the first illuminated region at the target site.

According to further examples, the adjustment device may be an opto-electric device, an opto-acoustic device, or a mechanical device. The adjustment device may also be a Pockels cell device. The Pockels cell device may include a crystal mixture of potassium tantalate and potassium niobate (KTN). The adjustment device may be configured to oscillate the laser beam with a sinusoidal variation in a first direction. The adjustment device may also be configured to oscillate the laser beam by deflecting the laser beam to point from the first location to the second location. The system may further include another adjustment device configured to oscillate the laser beam with a sinusoidal variation in a second direction that is orthogonal to the first direction. The system may also include a focus device configured to focus the adjusted laser beam. The target site may be a portion of a retinal pigment epithelium (RPE) layer. To create the second illuminated region at the second location of the target site, the adjustment device may be configured to oscillate the laser beam to achieve a total deflection angle of less than 0.1 mrad for a deflector—RPE layer distance of greater than 10 centimeters. To create the second illuminated region at the second location of the target site, the adjustment device may be configured to oscillate the laser beam at a frequency of 0.6 MHz or greater. The laser may be configured to generate the laser beam for a duration of less than two microseconds. The controller may be further configured to control the operation of the adjustment device such that the combined illuminated region is formed at a portion of the target site that has a diameter of less than 70 micrometers. The controller may be further configured to use the combined illuminated region to generate heat at the target site for treatment.

According to further examples, another laser treatment system is provided. The system may include a laser configured to generate a laser beam to create a first illuminated region at a first location of a target site and generate heat at the target site through the laser beam and an electro-optic device having an adjustable refractive index medium, coupled to the laser, and configured to oscillate the laser beam in a first direction to create a second illuminated region at a second location of the target site. The system may further include a controller coupled to the laser and the electro-optic device and configured to control an operation of the electro-optic device such that the first and second regions at least partially overlap to form a combined illuminated region at the target site, where a uniformity of illumination of the combined illuminated region at the target site is greater than a uniformity of illumination of the first illuminated region at the target site.

According to some examples, the electro-optic device is configured to oscillate the laser beam by deflecting the laser beam to point from the first location to the second location. The electro-optic device may be a Pockels cell device. The Pockels cell device may include a crystal mixture of potassium tantalate and potassium niobate (KTN). The system may further include another electro-optic device configured to oscillate the laser beam with a sinusoidal variation in a second direction that is orthogonal to the first direction. The system may also include a focus device configured to focus the adjusted laser beam. The target site may be a portion of a retinal pigment epithelium (RPE) layer. To create the second illuminated region at the second location of the target site, the electro-optic device may be configured to oscillate the laser beam to achieve a total deflection angle of less than 0.1 mrad for a deflector—RPE layer distance of greater than 10 centimeters. To create the second illuminated region at the second location of the target site, the electro-optic device may be configured to oscillate the laser beam at a frequency of 0.6 MHz or greater. The laser may be configured to generate the laser beam for a duration of less than two microseconds. The controller may be further configured to control the operation of the electro-optic device such that the combined illuminated region is formed at a portion of the target site that has a diameter of less than 70 micrometers.

According to yet further examples, a method for reducing laser illumination variability is provided. The method may include generating a laser beam directed at a substrate, where the laser beam creates a first illumination variation on the substrate. The method may further include deflecting the laser beam to create a second illumination variation on the substrate, where a combined illumination variation formed from the first and second illumination variations has a lower average variation than either the first or second illumination variations. The method may further include using the laser beam forming the combined illumination variation to generate heat on the substrate.

According to other examples, a heating uniformity of the laser beam forming the combined illumination variation may be greater than a heating uniformity of the laser beam creating the first illumination variation and a heating uniformity of the laser beam creating the second illumination variation. Deflecting the laser beam to create the second illumination variation on the substrate may include deflecting the laser beam through one or more of an opto-electric interference, an opto-acoustic interference, or a mechanical interference. Deflecting the laser beam to create the second illumination variation on the substrate may include deflecting the laser beam to achieve a total deflection angle of less than 0.1 mrad for a deflector—substrate distance of greater than 10 centimeters; and generating the laser beam for a duration of less than two microseconds.

There are various vehicles by which processes and/or systems and/or other technologies described herein may be effected (e.g., hardware, software, and/or firmware), and that the preferred vehicle may vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, each function and/or operation within such block diagrams, flowcharts, or examples may be implemented, individually and/or collectively, by a wide range of hardware, software, firmware, or virtually any combination thereof. In one embodiment, several portions of the subject matter described herein may be implemented via application specific integrated circuits (ASICs), field programmable gate arrays (FPGAs), digital signal processors (DSPs), or other integrated formats. However, some aspects of the embodiments disclosed herein, in whole or in part, may be equivalently implemented in integrated circuits, as one or more computer programs executing on one or more computers (e.g., as one or more programs executing on one or more computer systems), as one or more programs executing on one or more processors (e.g., as one or more programs executing on one or more microprocessors), as firmware, or as virtually any combination thereof, and that designing the circuitry and/or writing the code for the software and/or firmware is possible in light of this disclosure.

The present disclosure is not to be limited in terms of the particular embodiments described in this application, which are intended as illustrations of various aspects. Many modifications and variations can be made without departing from its spirit and scope. Functionally equivalent methods and apparatuses within the scope of the disclosure, in addition to those enumerated herein, are possible from the foregoing descriptions. Such modifications and variations are intended to fall within the scope of the appended claims. The present disclosure is to be limited only by the terms of the appended claims, along with the full scope of equivalents to which such claims are entitled. The terminology used herein is for the purpose of describing particular embodiments only, and is not intended to be limiting.

In addition, the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive (HDD), a compact disc (CD), a digital versatile disk (DVD), a digital tape, a computer memory, a solid state drive (SSD), etc.; and a transmission type medium such as a digital and/or an analog communication medium (e.g., a fiber optic cable, a waveguide, a wired communication link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein may be integrated into a data processing system via a reasonable amount of experimentation. A data processing system may include one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity of gantry systems; control motors to move and/or adjust components and/or quantities).

A data processing system may be implemented utilizing any suitable commercially available components, such as those found in data computing/communication and/or network computing/communication systems. The herein described subject matter sometimes illustrates different components contained within, or connected with, different other components. Such depicted architectures are merely exemplary, and, in fact, many other architectures may be implemented which achieve the same functionality. In a conceptual sense, any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermediate components. Likewise, any two components so associated may also be viewed as being “operably connected”, or “operably coupled”, to each other to achieve the desired functionality, and any two components capable of being so associated may also be viewed as being “operably couplable”, to each other to achieve the desired functionality. Specific examples of operably couplable include but are not limited to physically connectable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

Furthermore, in those instances where a convention analogous to “at least one of A, B, and C, etc.” is used, in general, such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. All language such as “up to,” “at least,” “greater than,” “less than,” and the like include the number recited and refer to ranges which can be subsequently broken down into subranges as discussed above. Finally, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments are possible. The various aspects and embodiments disclosed herein are for purposes of illustration and are not intended to be limiting, with the true scope and spirit being indicated by the following claims.

1. A method for laser treatment with increased illumination uniformity, the method comprising:
generating a laser beam to create a first illuminated region at a first location of a target site;
adjusting the laser beam by oscillating the laser beam through an opto-electric interference to create a second illuminated region at a second location of the target site, such that:
the first and second regions at least partially overlap to form a combined illuminated region at the target site;
a uniformity of illumination of the combined illuminated region at the target site is greater than a uniformity of illumination of the first illuminated region at the target site; and
performing treatment at the target site using the combined illuminated region of the laser beam.

2. The method of claim 1, wherein adjusting the laser beam to create the second illuminated region at the second location of the target site further comprises:
oscillating the laser beam through one or more of an opto-acoustic interference or a mechanical interference.

3. The method of claim 2, wherein oscillating the laser beam comprises:
deflecting the laser beam to point from the first location to the second location.

4. The method of claim 1, wherein oscillating the laser beam through the opto-electric interference comprises:
oscillating the laser beam through an electro-optic device having an adjustable refractive index medium or generating a sinusoidal variation in the laser beam through a Pockels cell device.

5. (canceled)

6. The method of claim 1, wherein generating the laser beam to create the first illuminated region at the first location of the target site comprises:
creating the first illuminated region at a portion of a retinal pigment epithelium (RPE) layer.

7. (canceled)

8. (canceled)

9. The method of claim 1, wherein generating the laser beam to create the first illuminated region at the first location of the target site comprises:
generating the laser beam for a duration of less than two microseconds.

10. (canceled)

11. The method of claim 1, further comprising:
sweeping the adjusted laser beam in two orthogonal directions.

12. (canceled)

13. (canceled)

14. A laser treatment system comprising:
a laser configured to generate a laser beam to create a first illuminated region at a first location of a target site;
an adjustment device coupled to the laser and configured to adjust the laser beam to create a second illuminated region at a second location of the target site by oscillating the laser beam through an opto-acoustic interference; and
a controller coupled to the laser and the adjustment device, the controller configured to control an operation of the adjustment device such that the first and second regions at least partially overlap to form a combined illuminated region at the target site and a uniformity of illumination of the combined illuminated region at the target site is greater than a uniformity of illumination of the first illuminated region at the target site.

15.-17. (canceled)

18. The system of claim 14, wherein the adjustment device is configured to oscillate the laser beam with a sinusoidal variation in a first direction or by deflecting the laser beam to point from the first location to the second location.

19. (canceled)

20. The system of claim 18, further comprising another adjustment device configured to oscillate the laser beam with a sinusoidal variation in a second direction that is orthogonal to the first direction.

21. The system of claim 14, further comprising a focus device configured to focus the adjusted laser beam.

22. The system of claim 14, wherein the target site is a portion of a retinal pigment epithelium (RPE) layer.

23. The system of claim 22, wherein, to create the second illuminated region at the second location of the target site, the adjustment device is configured to oscillate the laser beam to achieve a total deflection angle of less than 0.1 mrad for a deflector—RPE layer distance of greater than 10 centimeters.

24. The system of claim 14, wherein, to create the second illuminated region at the second location of the target site, the adjustment device is configured to oscillate the laser beam at a frequency of 0.6 MHz or greater.

25. The system of claim 14, wherein the laser is configured to generate the laser beam for a duration of less than two microseconds.

26. (canceled)

27. (canceled)

28. A laser treatment system comprising:
a laser configured to generate a laser beam to create a first illuminated region at a first location of a target site and generate heat at the target site through the laser beam;
an electro-optic device comprising an adjustable refractive index medium, the electro-optic device coupled to the laser and configured to oscillate the laser beam in a first direction to create a second illuminated region at a second location of the target site; and
a controller coupled to the laser and the electro-optic device, the controller configured to control an operation of the electro-optic device such that the first and second regions at least partially overlap to form a combined illuminated region at the target site and a uniformity of illumination of the combined illuminated region at the target site is greater than a uniformity of illumination of the first illuminated region at the target site.

29. (canceled)

30. The system of claim 28, wherein the electro-optic device is a Pockels cell device.

31. The system of claim 30, wherein the Pockels cell device comprises a crystal mixture of potassium tantalate and potassium niobate (KTN).

32. The system of claim 31, further comprising another electro-optic device configured to oscillate the laser beam with a sinusoidal variation in a second direction that is orthogonal to the first direction.

33.-35. (canceled)

36. The system of claim 28, wherein, to create the second illuminated region at the second location of the target site, the electro-optic device is configured to oscillate the laser beam at a frequency of 0.6 MHz or greater.

37.-42. (canceled)
